(19) United States
(12) Patent Application Publication
(10) Pub. No.: US 2022/0378999 A1
(43) Pub. Date: Dec. 1, 2022

(54) PERCUTANEOUS CATHETER AND METHOD OF USING PERCUTANEOUS CATHETER

(71) Applicant: TERUMO KABUSHIKI KAISHA, TOKYO (JP)

(72) Inventor: Kenji Yokoyama, Kanagawa (JP)

(21) Appl. No.: 17/884,932

(22) Filed: Aug. 10, 2022

Related U.S. Application Data
(63) Continuation of application No. PCT/JP2021/007349, filed on Feb. 26, 2021.

(30) Foreign Application Priority Data
Mar. 5, 2020 (JP) 2020-038150

(51) Int. Cl. A61M 1/36; A61M 25/00; A61M 60/113; A61M 60/232; A61M 60/38

(52) U.S. Cl. CPC A61M 1/3659; A61M 25/005; A61M 25/007; A61M 60/113; A61M 60/232; A61M 60/38

(57) ABSTRACT

A catheter for percutaneous cardiopulmonary support has a lumen through which blood flows to a living body. The catheter comprises a catheter tube including a tubular reinforcing body including a plurality of wires braided into a mesh shape and a resin layer provided so as to cover the reinforcing body. A plurality of side holes are formed at a distal end of the catheter tube to communicate the lumen with the outside of the catheter tube. The side holes allow the blood flowing through the lumen to flow out in a direction intersecting an axial direction of the catheter tube when the catheter tube is indwelled. The plurality of side holes preferably may be spirally arranged in a circumferential direction of the tube.

### CROSS REFERENCE TO RELATED APPLICATIONS

This application is a continuation of PCT Application No. PCT/JP2021/007349, filed Feb. 26, 2021, based on and claiming priority to Japanese Application No. JP2020-038150, filed Mar. 5, 2020, both of which are incorporated herein by reference in their entirety.

### BACKGROUND OF THE INVENTION

The present invention relates to a percutaneous catheter and a method of using the percutaneous catheter.

Conventionally, treatment by percutaneous cardiopulmonary support (PCPS) has been performed in order to perform cardiopulmonary resuscitation, circulation assistance, and respiration assistance in emergency treatment. The percutaneous cardiopulmonary support is a method of temporarily assisting and substituting for a cardiopulmonary function using an extracorporeal circulation device.

The extracorporeal circulation device is provided with an extracorporeal circulation circuit formed of a centrifugal pump, an oxygenator, a blood removal path, a blood supply path and the like, and performs gas exchange on removed blood and supplies the blood to the blood supply path.

In the extracorporeal circulation circuit, a catheter (cannula) provided with a lumen through which the blood flows is used for the blood removal path and the blood supply path.

For example, JP 2015-165880A discloses a blood supply catheter and a blood removal catheter as a catheter used in an extracorporeal circulation circuit.

In general, a percutaneous catheter used in an extracorporeal circulation circuit is provided with a hole communicating with a lumen for removing or supplying blood. Especially, a hole of a blood supply catheter to be inserted into the femoral artery is formed by providing an opening at a distal end (most distal end) of the blood supply catheter. Therefore, most of blood sent out from the blood supply catheter flows to a distal end side of the blood supply catheter (for example, an abdominal aorta side) from the hole provided at the most distal end. Since the blood supply catheter is large with respect to a blood vessel into which this is inserted, there is a possibility that a blood flow of a lower limb on a proximal end side of the blood supply catheter is inhibited. Therefore, the lower limb cannot acquire an adequate blood amount and might cause lower limb ischemia.

### SUMMARY OF THE INVENTION

Therefore, an object of the present invention is to provide a percutaneous catheter capable of improving a blood flow in the lower limb on a proximal end side of the percutaneous catheter, and a method of using the percutaneous catheter.

A percutaneous catheter achieving the above-described object defines a lumen through which blood flows, and is comprised of a tube having a tubular reinforcing body including a plurality of wires braided into a mesh shape, a resin layer provided so as to cover the reinforcing body, and a side hole formed on a distal end part of the tube and communicating the lumen with an outside of the tube, in which the side hole allows the blood flowing through the lumen to flow out in a direction intersecting an axial direction of the tube when the tube is indwelled in a living body.

A method of using a percutaneous catheter achieving the above-described object includes steps of inserting the percutaneous catheter from a femoral artery side of one lower limb toward a femoral artery branch portion and indwelling in a living body, supplying blood from a side hole formed on a distal end part of the percutaneous catheter to a terminal end side of a femoral artery, which is a proximal end side of the percutaneous catheter, a femoral artery side of the other lower limb, and an abdominal aorta side, and removing the percutaneous catheter from an inside of the living body.

The percutaneous catheter formed as described above may allow the blood to flow out in the direction intersecting the axial direction of the tube and supply the blood toward both the distal end side and the proximal end side of the tube when indwelled in the living body. Therefore, the percutaneous catheter may improve a blood flow of the lower limb on the proximal end side of the percutaneous catheter.

According to the method of using the percutaneous catheter formed as described above, the percutaneous catheter may be inserted from one femoral artery side toward the femoral artery branch portion, and the percutaneous catheter indwelled in the living body may supply the blood toward the distal end side and the proximal end side of the tube. Therefore, the blood flow of the lower limb on the proximal end side of the percutaneous catheter may be improved.

### BRIEF DESCRIPTION OF THE DRAWINGS

FIG. 1 is a system diagram illustrating an example of an extracorporeal circulation device to which a percutaneous catheter according to an embodiment of the present invention is applied.

FIG. 2 is a side view illustrating a percutaneous catheter and a stylet according to an embodiment of the present invention.

FIG. 3 is an enlarged side view illustrating a distal end part of the percutaneous catheter according to the embodiment of the present invention.

FIG. 4 is a side cross-sectional view illustrating the percutaneous catheter according to the embodiment of the present invention.

FIG. 5 is a side view illustrating a state in which the stylet is inserted into the percutaneous catheter according to the embodiment of the present invention.

FIG. 6A is an enlarged view illustrating a side hole of the percutaneous catheter, and FIG. 6B is a cross-sectional view taken along line E-E of FIG. 6A.

FIG. 7 is a flowchart illustrating a method of using the percutaneous catheter.

FIG. 8 is a schematic diagram illustrating a method of using the percutaneous catheter.

### DETAILED DESCRIPTION OF PREFERRED EMBODIMENTS

Embodiments of the present invention are hereinafter described with reference to the accompanying drawings. The following description does not limit the technical scope or meaning of terms recited in claims. Dimensional ratios in the drawings are exaggerated for convenience of description, and might be different from actual ratios.

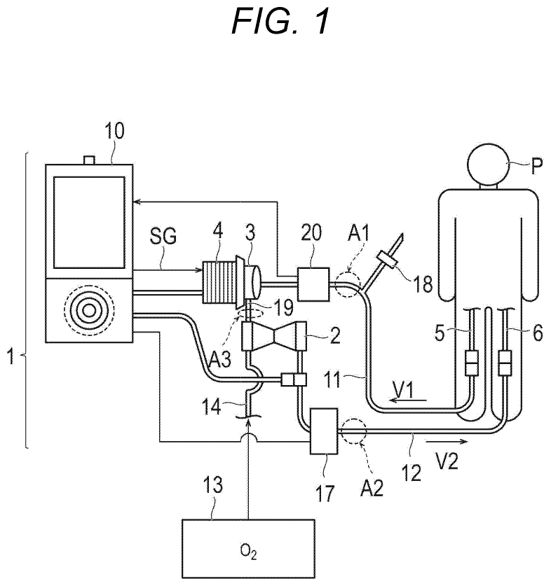

FIG. 1 is a system diagram illustrating an example of an extracorporeal circulation device 1 to which a percutaneous catheter according to the embodiment of the present invention is applied, the extracorporeal circulation device 1 used as percutaneous cardiopulmonary support (PCPS) that temporarily assists and substitutes for functions of the heart and lungs until a heart function recovers when the heart of a patient is weak.

According to the extracorporeal circulation device 1, it is possible to perform a veno-arterial (VA) procedure of removing blood from a vein (vena cava) of the patient by operating a pump, exchanging gas in the blood by an oxygenator 2 to oxygenate the blood, and then returning the blood to an artery (aorta) of the patient again. The extracorporeal circulation device 1 is a device that assists the heart and lungs. Hereinafter, a procedure of removing the blood from the patient, performing predetermined extracorporeal treatment, and then supplying the blood into the body of the patient again is referred to as “extracorporeal circulation”.

As illustrated in FIG. 1, the extracorporeal circulation device 1 includes a circulation circuit that circulates the blood. The circulation circuit includes an oxygenator 2, a centrifugal pump 3, a drive motor 4 as a drive means for driving the centrifugal pump 3, a vein-side catheter (percutaneous catheter for blood removal) 5, an artery-side catheter (blood supply catheter) 6, and a controller 10 as a control unit.

The vein-side catheter (blood removal catheter) 5 is inserted from the femoral vein, and a distal end of the vein-side catheter 5 is indwelled in the right atrium via the inferior vena cava. The vein-side catheter 5 is connected to the centrifugal pump 3 via a blood removal tube (blood removal line) 11. The blood removal tube 11 is a pipeline that sends the blood.

The artery-side catheter (blood supply catheter) 6 is inserted from the femoral artery.

When the drive motor 4 operates the centrifugal pump 3 by a command SG of the controller 10, the centrifugal pump 3 may remove the blood from the blood removal tube 11 and pass the blood via the oxygenator 2, then return the blood to a patient P via a blood supply tube (blood supply catheter) 12.

The oxygenator 2 is arranged between the centrifugal pump 3 and the blood supply tube 12. The oxygenator 2 performs gas exchange (oxygenation and/or carbon dioxide removal) on the blood. The oxygenator 2 is, for example, a membrane oxygenator, and a hollow fiber membrane oxygenator is especially preferably used. Oxygen gas is supplied from an oxygen gas supply unit 13 to the oxygenator 2 through a tube 14. The blood supply tube 12 is a pipeline that connects the oxygenator 2 to the artery-side catheter 6.

As the blood removal tube 11 and the blood supply tube 12, for example, a pipeline made of an elastically deformable and flexible synthetic resin having high transparency such as a vinyl chloride resin or silicone rubber may be used. In the blood removal tube 11, the blood as liquid flows in a V1 direction, and in the blood supply tube 12, the blood flows in a V2 direction.

In the circulation circuit illustrated in FIG. 1, an ultrasonic bubble detection sensor 20 is arranged in the middle of the blood removal tube 11. A fast clamp 17 is arranged in the middle of the blood supply tube 12.

In a case where a bubble is mixed in the circuit with the blood due to an erroneous operation of a three-way stopcock 18 or a breakage of the tube and the like during the extracorporeal circulation, then the ultrasonic bubble detection sensor 20 detects the mixed bubble. In a case where the ultrasonic bubble detection sensor 20 detects that there is the bubble in the blood sent in the blood removal tube 11, the ultrasonic bubble detection sensor 20 transmits a detection signal to the controller 10. On the basis of this detection signal, the controller 10 performs notification of a warning by an alarm, and decreases a rotation speed of the centrifugal pump 3 or stops the centrifugal pump 3. The controller 10 further instructs the fast clamp 17 to immediately close the blood supply tube 12 by the fast clamp 17. This prevents the bubble from being sent into the body of the patient P. In this manner, the controller 10 controls an operation of the extracorporeal circulation device 1 to prevent mixing of the bubble into the body of the patient P.

A tube 11 (or tube 12, 19) of the circulation circuit of the extracorporeal circulation device 1 is provided with a pressure sensor. For example, the pressure sensor may be attached to any one or all of an attachment position A1 of the blood removal tube 11, an attachment position A2 of the blood supply tube 12 of the circulation circuit, and an attachment position A3 of a connection tube 19 that connects the centrifugal pump 3 to the oxygenator 2. As a result, a pressure in the tube 11 (tube 12, 19) may be measured by the pressure sensor when the extracorporeal circulation is performed on the patient P by the extracorporeal circulation device 1. The attachment position of the pressure sensor is not limited to the attachment positions A1, A2, and A3 described above, and may be any position of the circulation circuit.

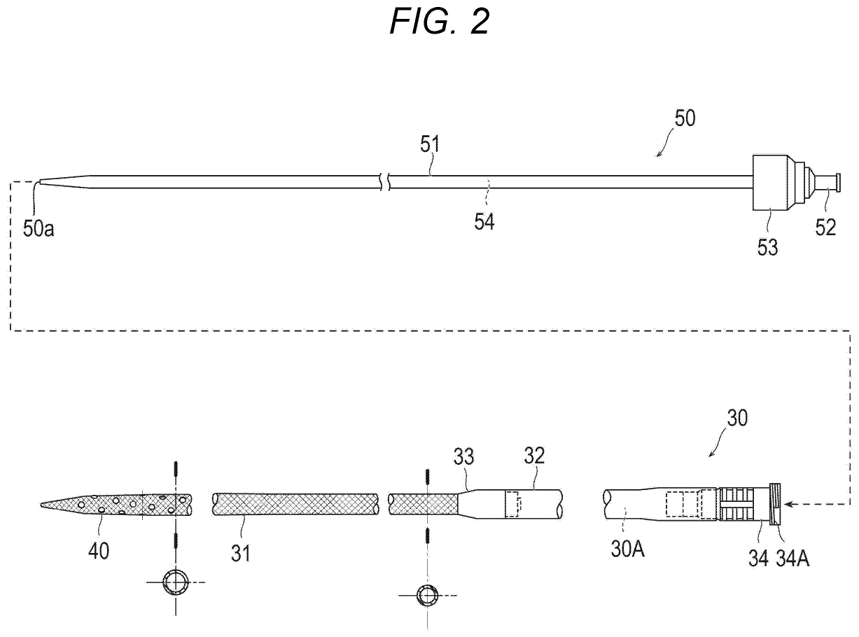

A percutaneous catheter (hereinafter, referred to as a “catheter”) 30 according to the embodiment of the present invention is described with reference to FIGS. 2 to 6. FIGS. 2 to 6 are views for describing a configuration of the catheter 30 according to this embodiment. The catheter 30 according to this embodiment is used as the artery-side catheter (blood supply catheter) 6 in FIG. 1.

As illustrated in FIG. 2, the catheter 30 according to this embodiment includes a catheter tube (corresponding to the “tube”) 31 provided with a side hole 40, a clamping tube 32 arranged on a proximal end side of the catheter tube 31, a catheter connector 33 that connects the catheter tube 31 to the clamping tube 32, and a lock connector 34.

In this specification, a side to be inserted into a living body is referred to as a “distal end” or a “distal end side”, and a hand side operated by an operator is referred to as a “proximal end” or a “proximal end side”. A distal end part means a certain range including the distal end (most distal end) and its periphery, and a proximal end part means a certain range including the proximal end (most proximal end) and its periphery.

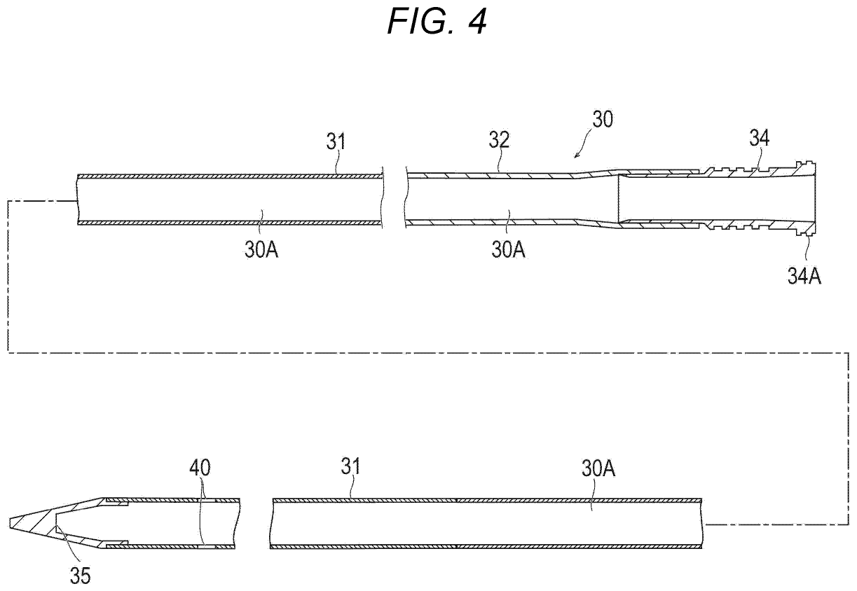

As illustrated in FIG. 4, the catheter 30 includes a lumen 30A penetrating from a distal end to a proximal end thereof. The side holes 40 communicating with the lumen 30A are arranged to be placed in different blood supply targets in the living body such that the blood may be efficiently removed.

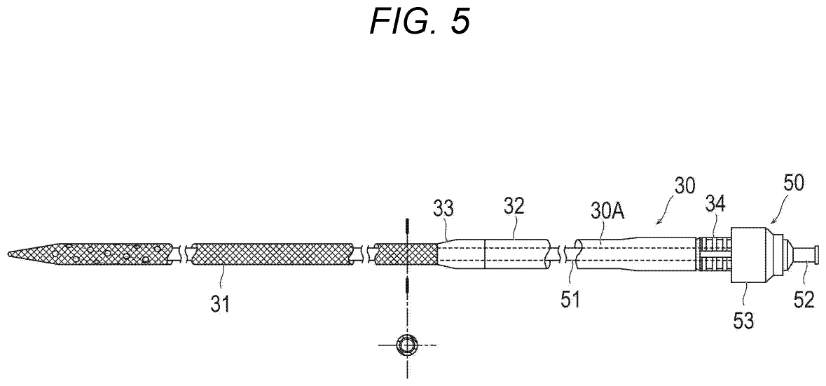

When the catheter 30 is inserted into the living body, a stylet 50 is used as illustrated in FIG. 5. The stylet 50 is inserted into the lumen 30A of the catheter 30, and the catheter 30 and the stylet 50 are inserted into the living body in a state of being integrated in advance. A method of using the catheter 30 is described later in detail.

Hereinafter, each configuration of the catheter 30 is described.

As illustrated in FIG. 2, the catheter 30 includes the catheter tube 31. A wall thickness of the catheter tube 31 is preferably made substantially constant. A length of the catheter tube 31 is set to a length necessary for arranging the side hole 40 provided on a distal end part of the catheter tube 31 in a desired blood supply target. The length of the catheter tube 31 may be set to, for example, 14 to 15 cm.

As illustrated in FIG. 2, the side hole 40 is a hole that penetrates a side surface of the catheter tube 31 except for a distal end (most distal end) and is opened to communicate with the lumen 30A of the catheter 30. The side hole 40 serves as a blood supply hole.

The side hole 40 may allow the blood to flow out in a direction intersecting the axial direction of the catheter tube 31 when the catheter tube 31 is indwelled in the blood vessel and the blood is supplied from the side hole 40. Therefore, the catheter tube 31 may generate a blood outflow from the side hole 40 that impacts a blood vessel wall. The blood that flows out of the side hole 40 and impacts the blood vessel wall may disperse a traveling direction of the blood flow toward the proximal end side and/or the distal end side of the catheter tube 31. The side hole 40 may send out the blood toward the proximal end side of the catheter tube 31 based on a pressure difference of the blood vessel. That is, an attractive force toward the distal end side of the catheter tube 31 is less likely to affect the blood flowing out of the catheter tube 31 in a case of supplying the blood from the side hole 40 of the catheter tube 31 than in a case of supplying the blood by providing the hole at the distal end of the catheter tube 31. Therefore, the side hole 40 may send out the blood not only toward the distal end side but also toward the proximal end side of the catheter tube 31.

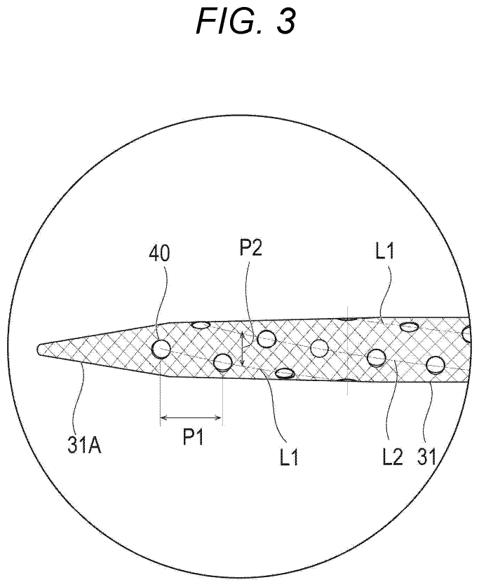

As illustrated in FIG. 3, a plurality of side holes 40 is preferably provided in the circumferential direction, and 24 side holes 40 are spirally arranged in the circumferential direction of the catheter tube 31. In this embodiment, the plurality of side holes 40 is arranged along a plurality of spirally-arranged virtual lines L1 and L2. At that time, one side hole 40 and another side hole 40 adjacent to the one side hole 40 arranged on one virtual line are arranged at different positions in the axial direction and circumferential direction of the catheter tube 31. Therefore, it is possible to suppress collision in the blood vessel between the blood sent out from one side hole 40 and the blood sent out from another side hole 40. Therefore, in the catheter 30 according to this embodiment, it is possible to suppress a possibility that the blood sent out from the plurality of side holes 40 from being stagnated in the blood vessel and to stably perform the blood supply.

The side hole 40 is a substantially circular hole, and a diameter thereof may be set to, for example, 1.0 to 1.5 mm. An interval P1 (refer to FIG. 3) between one side hole 40 and another side hole 40 may be set to, for example, 5 to 8 mm as seen in the axial direction of the catheter tube 31. The number and the diameter of the side holes 40 and the interval between one side hole 40 and another side hole 40 are not limited thereto, and may be appropriately set as necessary. The shape of the side hole 40 is not limited to a substantially circular shape, and may be a substantially elliptical shape, for example. Sizes of the plurality of side holes 40 may be the same or different from each other.

The virtual lines L1 and L2 (refer to FIG. 3) on which the plurality of side holes 40 is arranged are parallel to each other, and an interval P2 (refer to FIG. 3) may be set to, for example, 3 to 5 mm. The side hole 40 arranged on one virtual line (virtual line L2) is arranged so as to be located between two adjacent side holes 40 out of the side holes 40 arranged on the adjacent virtual line (virtual line L1) in the axial direction of the catheter tube 31. Note that “arranged between two adjacent side holes on the adjacent virtual line” means that the side hole 40 arranged on one virtual line may partially overlap the two side holes 40 arranged on the adjacent virtual line.

In this embodiment, it is described that the number of virtual lines on which the plurality of side holes 40 is arranged is two, but this is not especially limited. The interval between one virtual line and the adjacent virtual line is not especially limited, and may be appropriately set as necessary.

In this embodiment, the blood supply target is the femoral artery. The catheter 30 is inserted into the living body such that the side hole 40 is arranged in the femoral artery, and indwelled in the living body. Therefore, an inner diameter of the catheter tube 31 is preferably set to, for example, 4 to 6 mm. The wall thickness of the catheter tube 31 may be set to, for example, 0.3 to 0.5 mm.

As illustrated in FIG. 3, it is preferable that a distal end part of the catheter 30 forms a tapered portion 31A that gradually becomes thinner from the center of the catheter tube 31 toward the distal end part. A flat receiving surface 35 (refer to FIG. 4) that abuts a flat surface 50a of the stylet 50 used during the time when the catheter 30 is inserted into the living body is formed inside the distal end part of the catheter 30. The stylet 50 is described later in detail.

Hereinafter, a specific configuration of the catheter tube 31 is described.

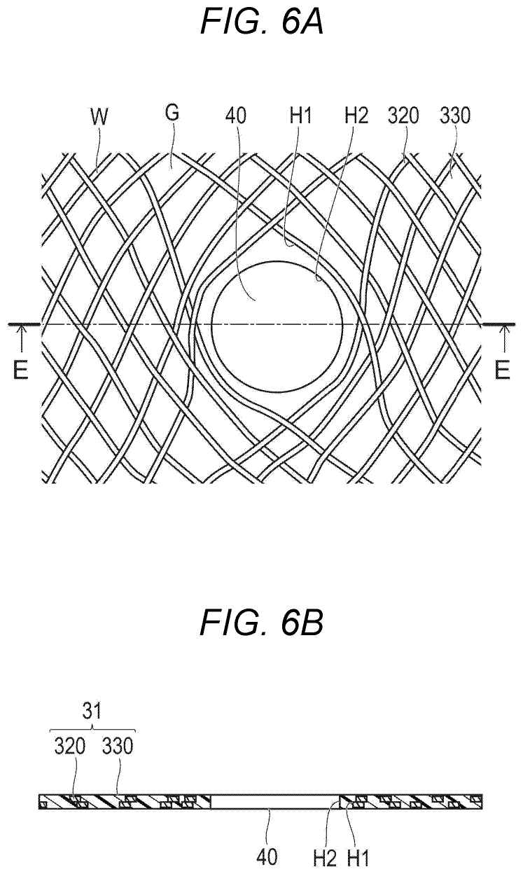

The catheter tube 31 includes a tubular reinforcing body 320 (refer to FIG. 6A) obtained by braiding wires W in a mesh shape so as to intersect, and a resin layer 330 (refer to FIG. 6B) provided so as to cover the reinforcing body 320.

As illustrated in FIG. 6A, the reinforcing body 320 is provided with a plurality of gaps G and a first opening H1 formed so as to have an opening area larger than that of the gap G in clearances between a plurality of wires W braided into a mesh shape.

Herein, the “opening area of the reinforcing body 320” means an area of a closed section surrounded by the plurality of wires W when the catheter 30 is seen from radially outside. In a portion in which the plurality of wires W overlap in a thickness direction, the innermost wire W forms the closed section.

The resin layer 330 is provided with a second opening H2 arranged so as to overlap the first opening H1 of the reinforcing body 320. The side hole 40 is formed in a portion where the first opening H1 and the second opening H2 overlap.

The wire W that forms the reinforcing body 320 is formed of a well-known shape memory material of shape memory metal or a shape memory resin. As the shape memory metal, for example, a titanium-based (Ni—Ti, Ti—Pd, Ti—Nb—Sn and the like) or copper-based alloy may be used. As the shape memory resin, for example, an acrylic resin, a transisoprene polymer, polynorbornene, a styrene-butadiene copolymer, and polyurethane may be used.

A cross-sectional shape of the wire W that forms the reinforcing body 320 is rectangular in this embodiment as illustrated in FIG. 6B, but is not limited thereto, and may be square, circular, elliptical and the like.

As a material forming the resin layer 330, a relatively soft known resin may be used, and for example, urethane, polyurethane, silicon, and vinyl chloride having low hardness may be used.

In a case where urethane and polyurethane are used, a hydrophilic coating may be applied to a surface thereof. As a result, since surface lubricity of the catheter tube 31 is improved, insertion into the living body is facilitated, operability is improved, and it is possible to prevent the blood vessel wall from being damaged. The blood and proteins are less likely to adhere, and it may be expected that thrombus formation is prevented.

The clamping tube 32 is provided on the proximal end side of the catheter tube 31. A lumen into which the stylet 50 may be inserted is provided inside the clamping tube 32. The clamping tube 32 may be formed using a material similar to that of the catheter tube 31.

The catheter connector 33 connects the catheter tube 31 to the clamping tube 32. A lumen into which the stylet 50 may be inserted is provided inside the catheter connector 33.

The lock connector 34 is connected to a proximal end side of the clamping tube 32. A lumen into which the stylet 50 may be inserted is provided inside the lock connector 34. A male screw portion 34A provided with a screw thread is provided on an outer surface on a proximal end side of the lock connector 34.

Next, a configuration of the stylet 50 is described.

As illustrated in FIG. 2, the stylet 50 includes a stylet tube 51 provided so as to extend in the axial direction, a stylet hub 52 to which a proximal end of the stylet tube 51 is fixed, and a screw ring 53 provided at a distal end of the stylet hub 52.

The stylet tube 51 is an elongated body extending in the axial direction and having relatively high rigidity. The stylet tube 51 is provided with a guidewire lumen 54 into which a guidewire (not illustrated) may be inserted. The stylet tube 51 is guided by the guide wire to be inserted into the living body together with the catheter 30. After the catheter 30 is indwelled in the living body, the stylet tube 51 is removed from the catheter 30 by pulling out the stylet hub 52 to the proximal end side.

A distal end part of the stylet tube 51 is provided with the flat surface 50a that abuts the receiving surface 35 (refer to FIG. 4) of the catheter tube 31. An outer diameter of the stylet tube 51 is made substantially the same as the inner diameter of the catheter tube 31, and a length of the stylet tube 51 is made substantially the same as the length of the catheter tube 31. The stylet tube 51 may transmit a pushing force to the distal end side by a hand side operation to the distal end part of the catheter tube 31. Therefore, when the flat surface 50a of the stylet tube 51 abuts the receiving surface 35 of the catheter tube 31, the stylet 50 may push the distal end of the catheter tube 31 toward the distal end side and is able to expand a narrow blood vessel.

The screw ring 53 includes a female screw portion (not illustrated) provided with a screw groove on an inner surface of an inner cavity. The stylet 50 may be attached to the catheter 30 by screwing the female screw portion of the screw ring 53 into the male screw portion 34A of the lock connector 34.

(Method of Using Catheter)

Next, a method of using the above-described catheter 30 is described.

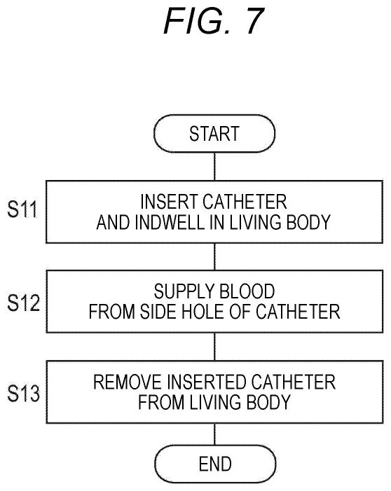

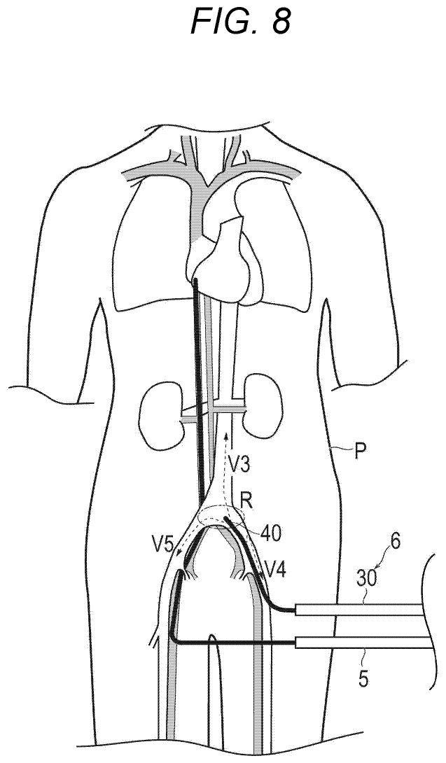

As illustrated in FIG. 7, the method of using the catheter 30 includes inserting the catheter tube 31 from a femoral artery side of one lower limb (left lower limb in this embodiment) toward a femoral artery branch portion R (FIG. 8) and indwelling the same in the living body (S11), supplying the blood from the side hole 40 formed on the distal end part of the catheter tube 31 to a terminal end side of the femoral artery, which is the proximal end side of the catheter tube 31, the femoral artery side of the other lower limb (right lower limb), and an abdominal aorta side (S12), and removing the catheter 30 from the inside of the living body (S13).

FIG. 2 illustrates a state before the stylet tube 51 of the stylet 50 is inserted into the lumen 30A of the catheter 30, and FIG. 5 illustrates a state after the stylet tube 51 is inserted into the lumen 30A of the catheter 30.

First, as illustrated in FIG. 5, the stylet tube 51 of the stylet 50 is inserted into the lumen 30A of the catheter 30. The stylet tube 51 passes through the inside of the catheter tube 31 and the flat surface 50a of the stylet tube 51 is arranged to abut the receiving surface 35 (refer to FIG. 4) of the catheter tube 31. At that time, a proximal end of the catheter 30 is fixed to the stylet hub 52 without stretching the catheter tube 31 by the stylet tube 51.

Next, the catheter 30 into which the stylet tube 51 is inserted is inserted from the femoral artery side of the left lower limb along the guide wire (not illustrated) which was previously inserted into a target site in the living body. The catheter 30 is inserted into the living body and indwelled there such that the distal end part of the catheter tube 31 is arranged at the femoral artery branch portion R (refer to FIG. 8) (S11). At that time, the plurality of side holes 40 is arranged in the femoral artery to be fed with the blood supply.

Next, the stylet tube 51 and the guide wire are removed from the catheter 30. At that time, the stylet tube 51 and the guide wire are temporarily pulled out to a site of the clamping tube 32 of the catheter 30 and clamped by forceps (not illustrated), and then completely removed from the catheter 30.

Next, the lock connector 34 of the catheter 30 is connected to the blood supply tube 12 of the extracorporeal circulation device 1 in FIG. 1. After confirming that the connection of the catheter on the blood removal side and the blood supply side is completed, the forceps of the clamping tube 32 are released to start the extracorporeal circulation.

At that time, the plurality of side holes 40 of the catheter tube 31 may allow the blood to flow out in a direction intersecting the axial direction of the catheter tube 31 to abut the blood vessel wall. Therefore, the side hole 40 may disperse the direction of sending out the blood by using the blood vessel wall. The side hole 40 may send out the blood toward the proximal end side of the catheter tube 31 using a pressure difference of the blood vessel. Therefore, the catheter 30 may supply the blood toward the abdominal aorta side (V3 direction), which is the distal end side of the catheter tube 31, the terminal end side of the femoral artery side (V4 direction), which is the proximal end side of the catheter tube 31, and the femoral artery side of the right lower limb (V5 direction) (S12).

In this embodiment, a ratio of the amount of blood flowing toward the terminal end side on the femoral artery side (V4 direction), which is the proximal end side of the catheter tube 31, to the amount of blood flowing toward the abdominal aorta side (V3 direction) is 0.1 to 0.2.

A ratio of the amount of blood flowing toward the femoral artery of the other lower limb (V5 direction) to the amount of blood flowing toward the abdominal aorta side (V3 direction) is 0.1 to 0.2.

When the extracorporeal circulation ends, the catheter 30 is removed from the blood vessel (S13) and hemostatic repair is performed by a surgical procedure as necessary at an insertion site.

As described above, the catheter (corresponding to the “percutaneous catheter”) 30 according to this embodiment is the percutaneous catheter provided with the lumen 30A through which the blood flows including the catheter tube (corresponding to the “tube”) 31 including the tubular reinforcing body 320 including the plurality of wires W braided into a mesh shape and the resin layer 330 provided so as to cover the reinforcing body 320, and the side hole 40 formed on the distal end part of the catheter tube 31 and communicating the lumen 30A with the outside of the catheter tube 31, and the side hole 40 allows the blood flowing through the lumen 30A to flow out in the direction intersecting the axial direction of the catheter tube 31 when the catheter tube 31 is indwelled in the living body.

According to the catheter 30 formed in this manner, the blood that flows out of the side hole 40 and hits the blood vessel wall may disperse the traveling direction toward the proximal end side or the distal end side of the catheter tube 31. The side hole 40 may send out the blood toward the proximal end side of the catheter tube 31 using a pressure difference of the blood vessel. Therefore, the blood flow of the lower limb on the proximal end side of the catheter 30 may be improved.

A plurality of side holes 40 is formed, and the plurality of side holes 40 is spirally arranged in the circumferential direction of the catheter tube 31. As a result, one side hole 40 and another side hole 40 adjacent to the one side hole 40 are arranged at different positions in the axial direction and circumferential direction of the catheter tube 31. Therefore, it is possible to suppress collision in the blood vessel between the blood sent out from one side hole 40 and the blood sent out from another side hole 40.

The method of using the catheter 30 according to this embodiment includes a step of inserting the catheter 30 from the femoral artery side of the left lower limb (corresponding to “one lower limb”) toward the femoral artery branch portion R and indwelling the same in the living body (S11), a step of supplying the blood from the side hole 40 formed at the distal end of the catheter 30 to the terminal end side of the femoral artery (V4 direction), which is the proximal end side of the catheter tube 31, the femoral artery side of the right lower limb (corresponding to the “other lower limb) (V5 direction), and the abdominal aorta side (V3 direction) (S12), and a step of removing the catheter 30 from the inside of the living body (S13). As a result, the side hole 40 may supply the blood toward the distal end side and the proximal end side of the catheter tube 31. Therefore, the blood flow of the lower limb on the proximal end side of the catheter 30 may be improved.

Although the percutaneous catheter and the method of using the percutaneous catheter according to the present invention are described above through the embodiment, the present invention is not limited to only each configuration described, and may be appropriately changed on the basis of the recitation in claims.

For example, although the plurality of side holes in this embodiment is spirally arranged in the circumferential direction of the catheter tube, an arrangement mode thereof is not especially limited as long as the effect of the present invention is exhibited, and they may be arranged linearly.

What is claimed is:

1. A percutaneous catheter provided with a lumen through which blood flows, the percutaneous catheter comprising:
a tubular reinforcing body including a plurality of wires braided into a mesh shape; and
a resin layer provided so as to cover the reinforcing body;
wherein the tubular reinforcing body and the resin layer define a tube with a plurality of side holes formed on a distal end part of the tube and communicating the lumen with an outside of the tube, wherein the plurality of side holes are spirally arranged in a circumferential direction of the tube; and
wherein the side holes allow the blood flowing through the lumen to flow out in a direction intersecting an axial direction of the tube when the tube is indwelled in a vessel of a living body.

2. The percutaneous catheter according to claim 1, wherein the plurality of side holes are arranged along a plurality of spirally-arranged virtual lines.

3. The percutaneous catheter according to claim 2 wherein the plurality of spirally-arranged virtual lines includes a first virtual line and a second virtual line which are arranged parallel to each other.

4. The percutaneous catheter according to claim 3, wherein the first and second virtual lines are spaced by an interval of between 3 mm and 5 mm.

5. The percutaneous catheter according to claim 3 wherein a respective side hole in the first virtual line is arranged so as to be located between two respective adjacent side holes arranged on the second virtual line in the axial direction of the catheter tube.

6. The percutaneous catheter according to claim 3 wherein one of the side holes in the first virtual line is spaced from an adjacent side hole in the first virtual line by an interval of between 5 mm to 8 mm in the axial direction of the tube.

7. A method of using a percutaneous catheter comprising the steps of:
inserting the percutaneous catheter from a femoral artery side of one lower limb toward a femoral artery branch portion and indwelling in a vessel of a living body;
supplying blood from a plurality of side holes formed at a distal end of the percutaneous catheter simultaneously to (1) a terminal end side of a femoral artery which is a proximal end side of the percutaneous catheter, (2) a femoral artery side of the other lower limb, and (3) an abdominal aorta, wherein the percutaneous catheter is comprised of a tubular reinforcing body and a resin layer defining a tube with the plurality of side holes formed on a distal end part of the tube and communicating the lumen with an outside of the tube, whereby the side holes allow the blood flowing through the lumen to flow out in a direction intersecting an axial direction of the tube; and
removing the percutaneous catheter from an inside of the living body.

8. The method of claim 7 wherein the plurality of side holes are spirally arranged in a circumferential direction of the tube.

9. The method according to claim 8, wherein the plurality of side holes are arranged along a plurality of spirally-arranged virtual lines.

10. The method according to claim 9 wherein the plurality of spirally-arranged virtual lines includes a first virtual line and a second virtual line which are arranged parallel to each other.

11. The method according to claim 9, wherein the first and second virtual lines are spaced by an interval of between 3 mm and 5 mm.

12. The method according to claim 9 wherein a respective side hole in the first virtual line is arranged so as to be located between two respective adjacent side holes arranged on the second virtual line in the axial direction of the catheter tube.

13. The method according to claim 9 wherein one of the side holes in the first virtual line is spaced from an adjacent side hole in the first virtual line by an interval of between 5 mm to 8 mm in the axial direction of the tube.
